(19) United States
(12) Patent Application Publication
(10) Pub. No.: US 2025/0387153 A1
(43) Pub. Date: Dec. 25, 2025

(54) MEDICAL TREATMENT TOOL

(71) Applicant: OLYMPUS CORPORATION, Tokyo (JP)

(72) Inventor: Takashi NAKAMURA, Tokyo (JP)

(73) Assignee: OLYMPUS CORPORATION, Tokyo (JP)

(21) Appl. No.: 19/316,212

(22) Filed: Sep. 2, 2025

Related U.S. Application Data
(63) Continuation of application No. 17/132,131, filed on Dec. 23, 2020, now Pat. No. 12,426,935.
Application No. 17/132,131 is a continuation of application No. PCT/JP2018/025379, filed on Jul. 4, 2018.

(51) Int. Cl. A61B 18/08; A61B 17/32; A61B 18/00

(52) U.S. Cl. CPC A61B 18/085; A61B 17/320092; A61B 2017/320093; A61B 2018/00101; A61B 2018/00595; A61B 2018/00607; A61B 2018/0063

(57) ABSTRACT

A medical treatment tool includes: a grasping portion having a first jaw and a second jaw; a plate-shaped blade that is provided in the first jaw and that has a thickness direction; a heater that supplies, to the first blade, thermal energy for treating a tissue grasped by the grasping portion; an energy insulating member that is arranged such that only an output surface of the blade, which comes into contact with the tissue, is exposed and that blocks leakage of the thermal energy to an outside of the first jaw; and first wiring that supplies electrical energy to the heater, wherein the blade, the heater, and the first wiring are arranged so as to be laminated in the thickness direction, and an area of a supply surface where the heater supplies the thermal energy to the blade is larger than an area of the output surface.

### CROSS-REFERENCE TO RELATED APPLICATIONS

This is continuation application of U.S. patent application Ser. No. 17/132,131 filed on Dec. 23, 2020, which is a continuation of International Application PCT/JP2018/025379, with an international filing date of Jul. 4, 2018, the entire contents of each of which are incorporated herein by reference.

### TECHNICAL FIELD

The present invention relates to a medical treatment tool.

### BACKGROUND ART

There is a known treatment tool in which tissue is sandwiched between a blade provided with a heater and a receiving member disposed at a position opposing the blade, and the tissue is sealed and incised as a result of heat being applied to the tissue from the blade heated by the heater (for example, see Patent Literature 1).

### CITATION LIST

### Patent Literature

{PTL 1} Japanese Unexamined Patent Application, Publication No. 2001-198137

### SUMMARY OF INVENTION

One aspect of the present invention is directed to a medical treatment tool comprising: a grasping portion having a first jaw and a second jaw that can be opened and closed; a plate-shaped first blade that is provided in the first jaw and that has a thickness direction; at least one heater that supplies, to the first blade, thermal energy for treating a tissue grasped by the grasping portion; an energy insulating member that is arranged such that only an output surface of the first blade, which comes into contact with the tissue, is exposed and that blocks leakage of the thermal energy to an outside of the first jaw; and first wiring that supplies electrical energy to the heater, wherein the first blade, the heater, and the first wiring are arranged so as to be laminated in the thickness direction, and an area of at least one supply surface where the heater supplies the thermal energy to the first blade is larger than an area of the output surface.

Another aspect of the present invention is directed to a medical treatment tool comprising: a grasping portion having a first jaw and a second jaw that can be opened and closed; a plate-shaped blade that is provided in the first jaw and that has a thickness direction; an energy supply portion that supplies, to the blade, treatment energy for treating a tissue grasped by the grasping portion; an energy insulating member that is arranged such that only an output surface of the blade, which comes into contact with the tissue, is exposed and that blocks leakage of the treatment energy to an outside of the first jaw; and first wiring that supplies electrical energy to the energy supply portion, wherein the blade, the energy supply portion, and the first wiring are arranged so as to be laminated in the thickness direction, and an area of a supply surface where the energy supply portion supplies the treatment energy to the blade is larger than an area of the output surface.

### BRIEF DESCRIPTION OF DRAWINGS

FIG. 1 is a front view partially showing a distal end portion of a medical treatment tool according to an embodiment of the present invention.

FIG. 2 is a cross-sectional view of a grasping portion showing the medical treatment tool in FIG. 1.

FIG. 3 is a cross-sectional view schematically showing the grasping portion in FIG. 2.

FIG. 4 is a perspective view showing an example of second wiring provided in the grasping portion in FIG. 2.

FIG. 5 is a schematic cross-sectional view of a grasping portion showing a first modification of the medical treatment tool in FIG. 1.

FIG. 6 is a schematic cross-sectional view of a grasping portion showing a second modification of the medical treatment tool in FIG. 1.

FIG. 7 is a schematic cross-sectional view of a grasping portion showing a third modification of the medical treatment tool in FIG. 1.

FIG. 8 is a schematic cross-sectional view of a grasping portion showing a fourth modification of the medical treatment tool in FIG. 1.

FIG. 9 is a schematic cross-sectional view of a grasping portion showing a fifth modification of the medical treatment tool in FIG. 1.

FIG. 10 is a schematic cross-sectional view of a grasping portion showing a sixth modification of the medical treatment tool in FIG. 1.

FIG. 11 is a schematic cross-sectional view of a grasping portion showing a seventh modification of the medical treatment tool in FIG. 1.

FIG. 12 is a schematic cross-sectional view of a grasping portion showing an eighth modification of the medical treatment tool in FIG. 1.

FIG. 13 is a schematic cross-sectional view of a grasping portion showing a ninth modification of the medical treatment tool in FIG. 1.

FIG. 14 is a schematic cross-sectional view of a grasping portion showing a tenth modification of the medical treatment tool in FIG. 1.

FIG. 15 is a schematic cross-sectional view of a grasping portion showing an eleventh modification of the medical treatment tool in FIG. 1.

### DESCRIPTION OF EMBODIMENT

A medical treatment tool 1 according to an embodiment of the present invention will be described below with reference to the drawings.

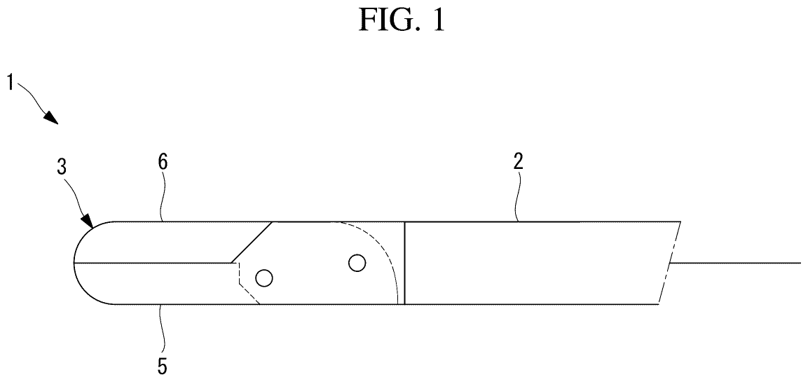

As shown in FIG. 1, the medical treatment tool 1 according to this embodiment is thermocoagulation incision forceps provided with: a grasping portion 3 at the distal end of an insertion portion 2 having a longitudinal axis; and an operating portion at the base end of the insertion portion 2.

The grasping portion 3 includes a first jaw 5 and a second jaw 6 that are coupled so as to be pivotable about an axis orthogonal to the longitudinal axis.

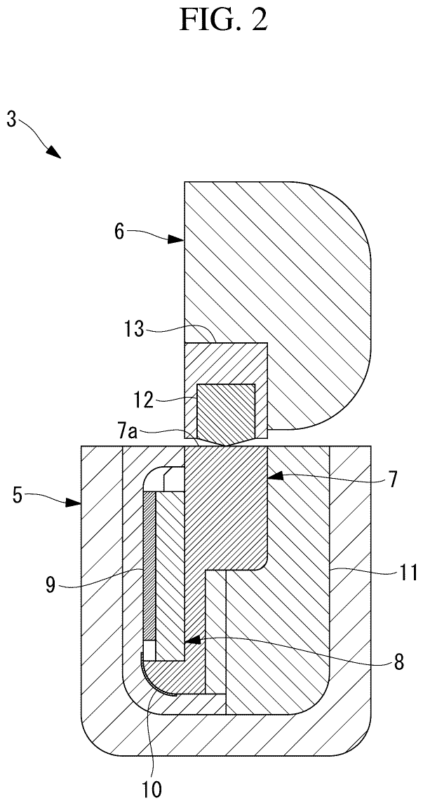

As shown in FIG. 2, the first jaw 5 includes: a blade 7 composed of a plate-shaped metal; a plate-shaped heater (energy supply portion, energy conversion portion) 8 that is in close contact with a surface of the blade 7; first wiring (energy supply portion, electrical-energy supply portion) 9 that supplies electrical energy to the heater 8; second wiring (electrical-energy supply portion) 10 that supplies a current to the blade 7; and a thermoelectrical insulating member (energy insulating member) 11 that exposes only one surface (output surface) 7a along the plate thickness direction of the blade 7 and covers other surfaces of the blade 7. The thermoelectrical insulating member 11 is composed of, for example, a resin material such as PEEK or LCP.

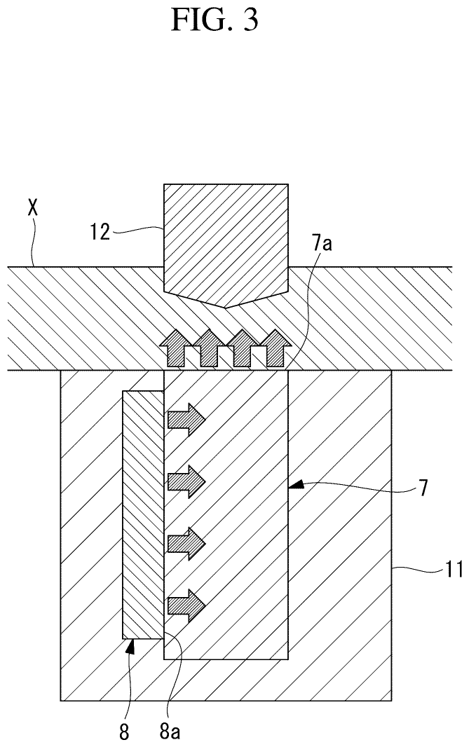

As shown in FIG. 3, the plate-shaped heater 8 is provided such that one surface (supply surface) 8a along the width direction is in close contact with one surface along the width direction of the blade 7. The area of the supply surface 8a is set to be sufficiently larger than the area of the output surface 7a of the blade 7.

The first wiring 9 is composed of a flexible wiring board (conductive member). The first wiring 9 is disposed at such a position that the first wiring 9 is in close contact with a flat surface along the width direction of the heater 8. By doing so, the blade 7, the heater 8, and the first wiring 9 are arranged in a laminated state in the thickness direction, whereby the overall thickness dimension is suppressed.

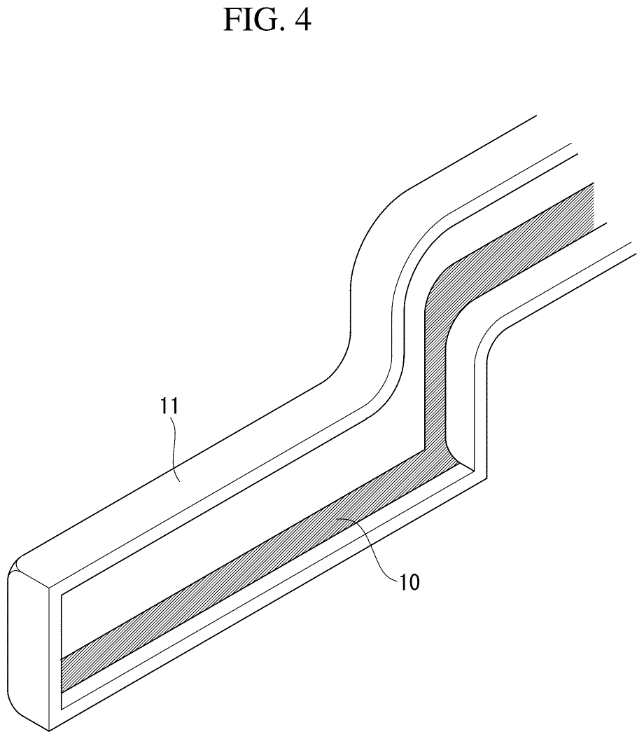

As shown in FIG. 4, the second wiring 10 is formed on an inner surface (surface) of the thermoelectrical insulating member 11 by means of, for example, the MID (Molded Interconnect Device) technology, and is composed of a conductive thin film that conducts the electrical energy.

As shown in FIG. 2, the second jaw 6 includes a blade 12 disposed at such a position that tissue X is sandwiched between the output surface 7a of the blade 7 in the first jaw 5 and the blade 12. A surface opposing the output surface 7a of the blade 7 in the first jaw 5 has a convex shape that protrudes most at the center in the width direction. The surfaces of the blade 12 in the second jaw 6, excluding the opposing surface, are also covered with a thermoelectrical insulating member 13.

The thermoelectrical insulating member 13 of the second jaw 6 has a shape in which the blade 12 is exposed in at least one direction intersecting the pivoting direction.

The operation of the thus-configured medical treatment tool 1 according to this embodiment will be described below.

With the medical treatment tool 1 according to this embodiment, the first jaw 5 and the second jaw 6 are pivoted relative to each other; in a state in which the output surface 7a of the blade 7 and the opposing surface of the blade 12 are separated from each other, tissue X is disposed between the output surface 7a and the opposing surface; and the output surface 7a and the opposing surface are brought close to each other, whereby the tissue X is sandwiched between the output surface 7a and the opposing surface.

In this state, when a current is supplied to the blade 7 via the second wiring 10, the blade 7 in the first jaw 5 functions as a monopolar electrode, supplying the current to the tissue X from the output surface 7a, and the tissue X in the vicinity of the blade 7 is cauterized and sealed.

Subsequently, electrical energy is supplied to the heater 8 via the first wiring 9, and the electrical energy is converted into thermal energy (treatment energy) by the heater 8. The converted thermal energy passes through the supply surface 8a of the heater 8 and is supplied to the blade 7, which is in close contact with the supply surface 8a, thereby heating the blade 7.

Because only the output surface 7a of the blade 7 is exposed and the surfaces other than the output surface 7a are covered with the thermoelectrical insulating member 11, the thermal energy supplied to the blade 7 is supplied to the tissue X via the output surface 7a. In this case, the output surface 7a of the blade 7 in the first jaw 5, which sandwiches the tissue X with the opposing surface of the blade 12 in the second jaw 6, has an area sufficiently smaller than that of the supply surface 8a where the thermal energy is supplied to the blade 7 from the heater 8; thus, the heat supplied to the blade 7 from the wide supply surface 8a of the heater 8 is output to the tissue X from the output surface 7a, which is narrower than the supply surface 8a, with an increased heat flux density.

In other words, leakage of the thermal energy from the blade 7 is suppressed by the thermoelectrical insulating member 11, and the thermal energy is supplied to the tissue X with an increased heat flux density; therefore, it is possible to effectively cut the tissue X. In addition, there is an advantage in that it is possible to reduce the size of the heater 8 that supplies the thermal energy for cutting the tissue X.

As described above, with the medical treatment tool 1 according to this embodiment, the plate-shaped heater 8 is laminated on the plate-shaped blade 7 in the plate thickness direction, and in addition, the first wiring 9 that supplies the electrical energy to the heater 8 is composed of a flexible wiring board and is laminated on the heater 8 in the plate thickness direction; therefore, it is possible to achieve a thickness reduction of the first jaw 5. Furthermore, the current is also supplied to the blade 7 by means of the second wiring 10, which is a conductive thin film formed by employing the MID technology; thus, the thermocoagulation incision forceps can be configured without sacrificing the thinness of the first jaw 5.

Furthermore, with the arrangement in which the area of the output surface 7a of the blade 7 is set to be smaller than the area of the supply surface 8a of the heater 8, there is an advantage in that it is possible to achieve a size reduction of the heater 8, in particular, the thickness reduction of the heater 8, and that the first jaw 5 can be configured to be even thinner.

In addition, the thermoelectrical insulating member 13 of the second jaw 6 has a shape in which the blade 12 is exposed in at least one direction intersecting the pivoting direction; thus, in a state in which the tissue X is sandwiched between the blade 7 in the first jaw 5 and the blade 12 in the second jaw 6, it is possible to visually recognize the blade 12 in the second jaw 6 from the outside. By doing so, there is an advantage in that it is possible to perform incision while confirming, in a more accurate manner, the position at which the tissue X is sandwiched.

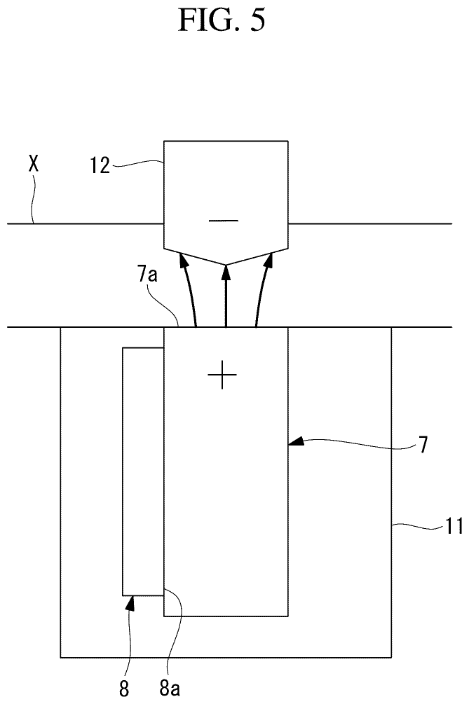

Note that, although the monopolar electrode structure in which only the blade 7 in the first jaw 5 is used as an electrode is employed in this embodiment, alternatively, as shown in FIG. 5, it is permissible to employ a bipolar electrode structure in which the blade 7 in the first jaw 5 is used as a first electrode, the blade 12 in the second jaw 6 is used as a second electrode, and a current is made to flow between the blades.

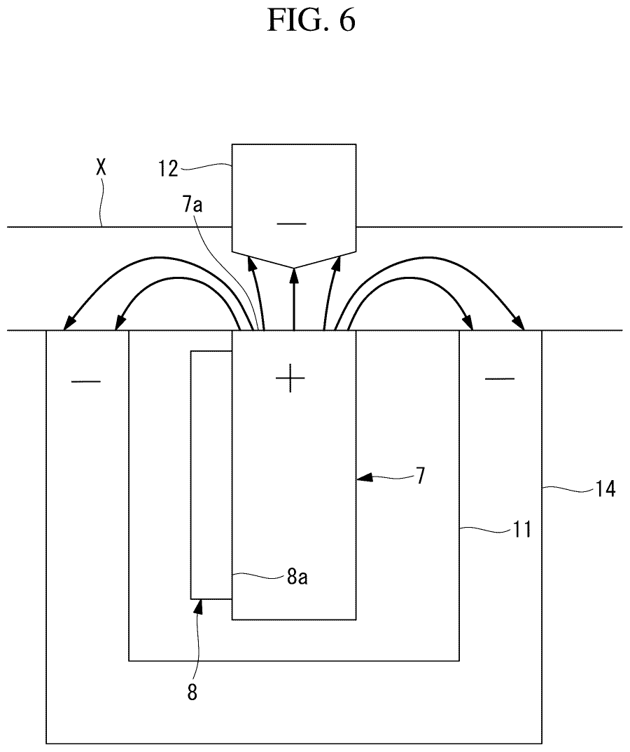

In addition, in this case, as shown in FIG. 6, third electrodes 14 may be arranged on both sides sandwiching, in the width direction, the blade 7 serving as the first electrode in the first jaw 5, with the thermoelectrical insulating member 11 interposed therebetween, and a current may also be made to flow between the blade 7 serving as the first electrode and the third electrodes 14. By doing so, there is an advantage in that it is possible to reliably perform sealing by expanding the cauterization range.

In addition, in this embodiment, the blade 12 in the second jaw 6 may be composed of an elastic material having a Young's modulus smaller than that of the blade 7 in the first jaw 5. By doing so, when the tissue X is grasped between the blades 7, 12, it is possible to prevent the problem of the tissue X being unintentionally cut due to the grasping force.

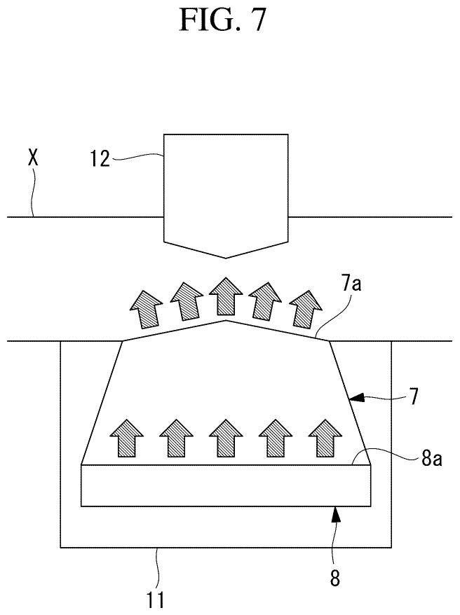

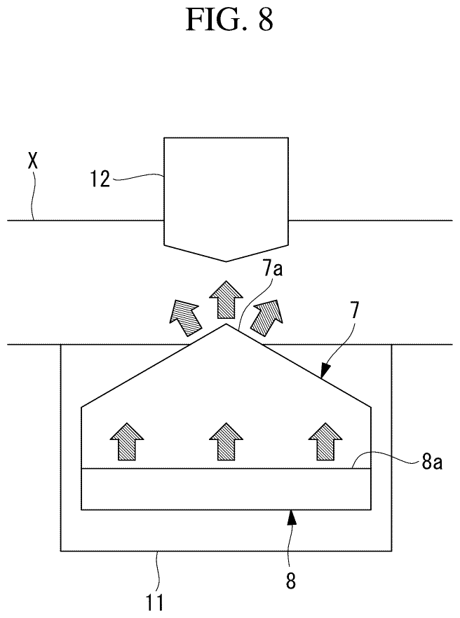

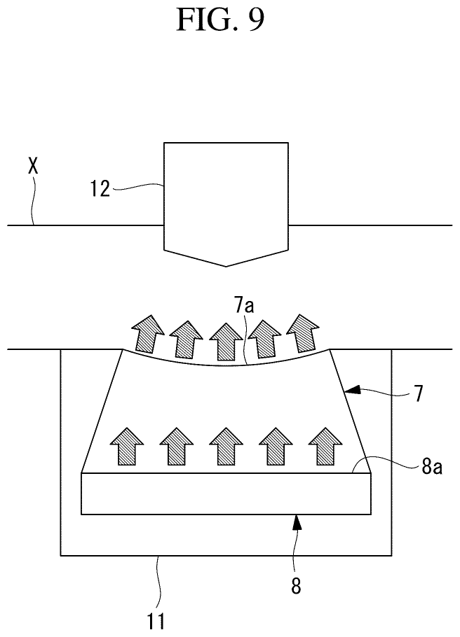

In addition, although the output surface 7a of the blade 7 in the first jaw 5 is configured to have a planar shape in this embodiment, alternatively, a surface shape of any form may be employed. For example, the output surface 7a may be formed in a convex shape as shown in FIGS. 7 and 8, or may be configured to be a concave surface as shown in FIG. 9. By configuring the output surface 7a to be a concave surface, there is an advantage in that diffusion of the heat flux can be further suppressed.

In addition, the blade 7 in the first jaw 5 and the heater 8 are configured to have a flat plate shape and are arranged in a laminated state such that the width direction of the first jaw 5 and the plate thickness direction of the blade 7 and the heater 8 are aligned in this embodiment; however, alternatively, the arrangement of the blade 7 and the heater 8 may be changed arbitrarily, as shown in FIGS. 7 and 8, so long as the area of the output surface 7a is smaller than the area of the supply surface 8a where the thermal energy is supplied from the heater 8.

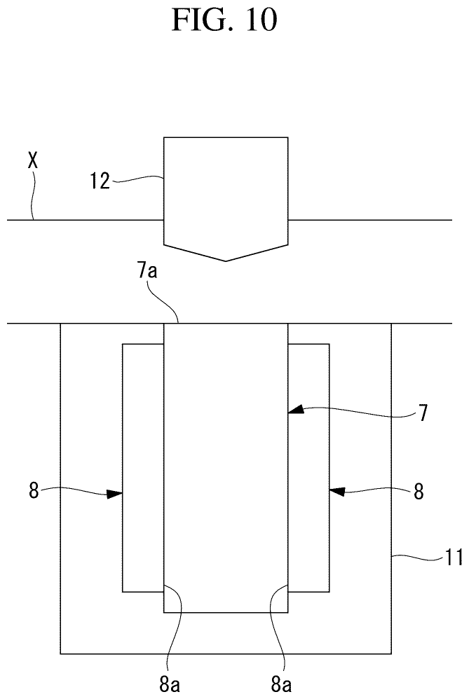

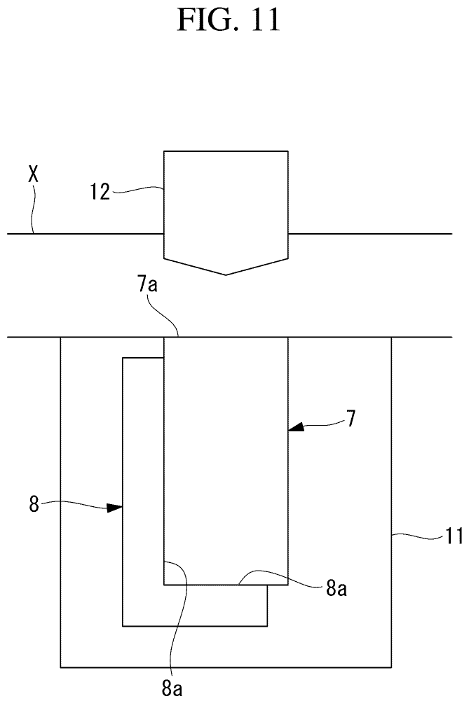

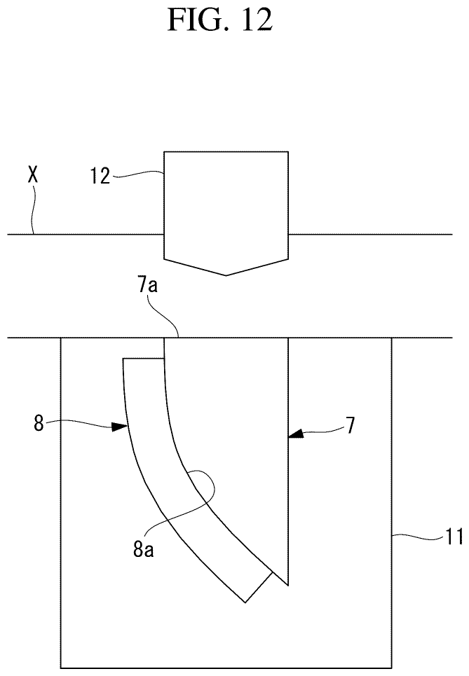

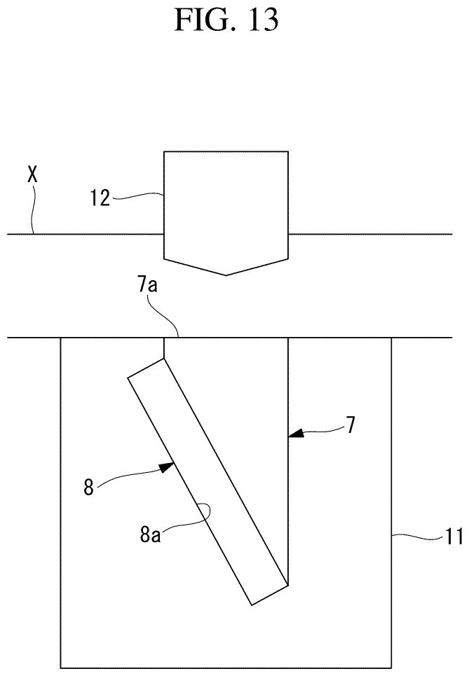

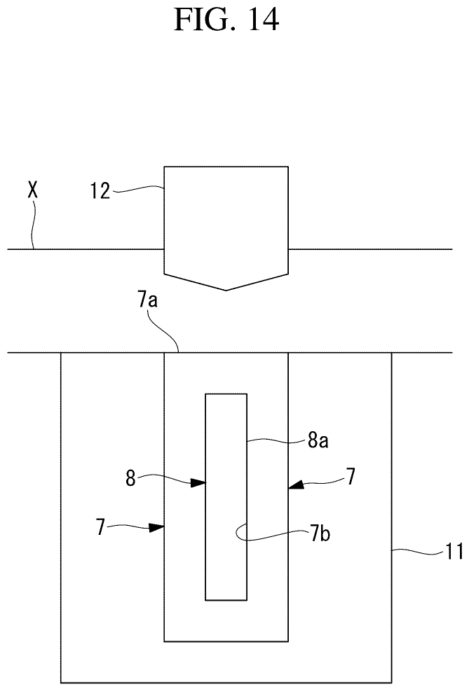

In addition, although one surface of the heater 8 is set to be the supply surface 8a in the example shown in FIG. 2, alternatively, as shown in FIGS. 10 and 11, a plurality of supply surfaces 8a may be provided, or the heater 8 may be brought into contact with a plurality of surfaces of the blade 7. In addition, as shown in FIGS. 12 and 13, the surface of the blade 7 with which the supply surface 8a is brought into close contact may be configured to be a curved surface or an inclined surface to ensure a larger area for the supply surface 8a. In addition, as shown in FIG. 14, the entire peripheral surface of the heater 8 may be set to be the supply surface 8a by fitting the heater 8 into a hole 7b formed in the blade 7.

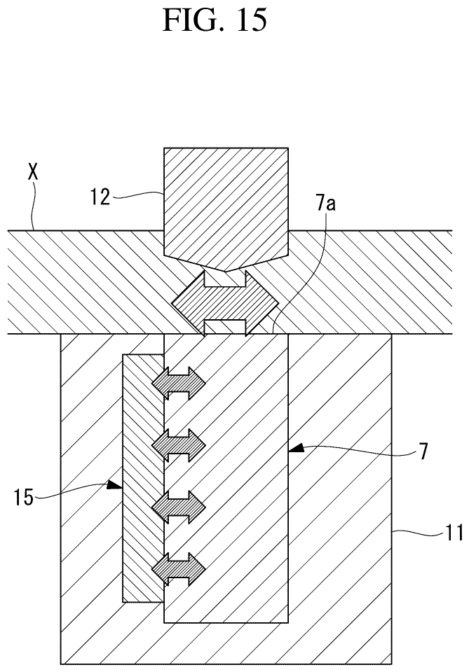

In addition, although the heater 8 that converts the electrical energy into the thermal energy is employed as the energy conversion portion in this embodiment, alternatively, as shown in FIG. 15, an ultrasonic vibrator 15 may be employed as the energy conversion portion, and ultrasonic waves may be supplied to the tissue X as the treatment energy.

As a result, the above-described embodiment leads to the following aspect.

An aspect of the present invention is directed to a medical treatment tool including: a grasping portion having a first jaw and a second jaw that can be opened and closed; blades that are respectively provided in the first jaw and the second jaw; and energy supply portions that supply, to the blade in the first jaw, treatment energy for treating tissue sandwiched between the blades, wherein the first jaw includes an energy insulating member that covers the blade in the first jaw to block leakage of the treatment energy to the outside of the first jaw, the energy insulating member is disposed at such a position that only an output surface, which comes into contact with the tissue sandwiched between the blades in the first jaw and the second jaw, is exposed, and the area of a supply surface where the energy supply portion supplies the treatment energy to the first jaw is larger than the area of the output surface.

With this aspect, tissue is disposed between the first jaw and the second jaw of the grasping portion, and by closing the first jaw and the second jaw, the tissue is grasped in a state in which the tissue is sandwiched between the first jaw and the second jaw. In this state, when the energy supply portion supplies the treatment energy to the blade in the first jaw, the treatment energy is supplied to the tissue via the blade, whereby treatment of the tissue can be performed.

The blade in the first jaw is covered with the energy insulating member and only the output surface that comes into contact with the tissue is exposed; thus, the treatment energy is supplied to the tissue via the output surface. In addition, the area of the supply surface where the energy supply portion supplies the treatment energy to the blade is set to be larger than the area of the output surface; therefore, the treatment energy supplied to the blade from the supply surface having a large area is supplied to the tissue in a state in which the energy density is increased when passing through the output surface having a small area. By doing so, it is possible to effectively incise the tissue while reducing the width of the blade in the first jaw sandwiching the tissue together with the blade in the second jaw.

In the abovementioned aspect, the energy supply portion may include: an electrical-energy supply portion that supplies electrical energy; and an energy conversion portion that converts the supplied electrical energy into the treatment energy.

With this configuration, the electrical energy supplied from the electrical-energy supply portion is converted into the treatment energy by the energy conversion portion, and the treatment energy is supplied to the blade in the first jaw.

In the abovementioned aspect, the energy conversion portion may be formed in a flat plate shape having the supply surface on one surface thereof.

With this configuration, the wide supply surface of the plate-shaped energy conversion portion is brought into contact with the blade, and thus, it is possible to supply a greater amount of the treatment energy to the blade while the cross-sectional shape of the first jaw is kept small.

In the abovementioned aspect, the blade in one of the first jaw and the second jaw may include a first electrode that supplies a current to the tissue sandwiched between said blade and the other blade.

With this configuration, the tissue can be sealed by supplying a current to the tissue from the first electrode in a state in which the tissue is sandwiched between the blades in the first jaw and the second jaw.

In the abovementioned aspect, the other blade may include a second electrode that makes a current flow between the first electrode and the second electrode.

With this configuration, by supplying a current to the tissue from the first electrode in a state in which the tissue is sandwiched between the blades in the first jaw and the second jaw, the current supplied to the tissue flows to the second electrode provided in the other blade. By doing so, the tissue can be sealed in a more reliable manner by means of the bipolar system.

In the abovementioned aspect, the first jaw may include: the first electrode; and third electrodes that are arranged on both sides sandwiching the first electrode in a width direction, with an electrical insulating material interposed therebetween, and that make a current flow between the first electrode and the third electrodes.

With this configuration, when a current is supplied to the tissue from the first electrode provided in the blade in the first jaw in a state in which the tissue is sandwiched between the blades in the first jaw and the second jaw, the current flows in an area between the second electrode provided in the second jaw and the third electrodes in the first jaw. Thus, the tissue can be sealed over a wider range.

In the abovementioned aspect, the energy insulating member may be composed of an electrical insulating material, and the electrical-energy supply portion may be formed on a surface of the energy insulating member and may include a conductive thin film that conducts the electrical energy.

With this configuration, it is possible to electrically insulate the blade in the first jaw from the outside by means of the energy insulating member, and to supply the electrical energy to the blade by means of the conductive thin film provided on the surface of the energy insulating member. By doing so, the first jaw can be made thinner.

In the abovementioned aspect, the electrical-energy supply portion may be provided so as to be laminated in a thickness direction of the energy conversion portion, and may include a thin plate-like conductive member that conducts the electrical energy.

With this configuration, the first jaw can be made thinner.

In the abovementioned aspect, the energy conversion portion may have a plurality of the supply surfaces.

With this configuration, it is possible to ensure wide supply surfaces, thereby supplying a greater amount of the treatment energy to the blade, while suppressing the maximum size of the energy conversion portion.

In the abovementioned aspect, the second jaw may have a shape in which the blade is exposed in at least one direction intersecting the opening/closing direction.

With this configuration, it is possible to enhance the visibility of the position of the blade relative to the tissue when the first jaw and the second jaw are closed.

In the abovementioned aspect, the output surface may be a flat surface or a concave surface.

With this configuration, it is possible to suppress diffusion of the treatment energy output from the output surface, thereby efficiently supplying the treatment energy to the tissue.

In the abovementioned aspect, the blade in the second jaw may be made of a material having a Young's modulus smaller than that of the blade in the first jaw.

With this configuration, when the tissue is sandwiched between the first jaw and the second jaw, it is possible to prevent an excessive grasping force from being applied to the tissue by elastically deforming the blade, thereby preventing the tissue from being cut before sealing.

The present invention affords an advantage in that it is possible to effectively incise tissue while reducing the width of a blade sandwiching the tissue.

### REFERENCE SIGNS LIST

1 medical treatment tool
3 grasping portion
5 first jaw
6 second jaw
7, 12 blade (first electrode, second electrode)
7a output surface
8 heater (energy supply portion, energy conversion portion)
8a supply surface
9 first wiring (energy supply portion, electrical-energy supply portion, conductive member)
10 second wiring (electrical-energy supply portion, conductive thin film)
11, 13 thermoelectrical insulating member (energy insulating member, electrical insulating material)
14 third electrode
15 ultrasonic vibrator (energy supply portion, energy conversion portion)
X tissue

What is claimed is:

1. A medical treatment tool comprising:
a grasping portion having a first jaw and a second jaw configured to be opened and closed relative to the first jaw in an opening and closing direction,
wherein the first jaw comprises:
an electric heater having a supply surface, the electric heater configured to supply thermal energy to a first blade; and
the first blade having:
an output surface facing the second jaw; and
a heat receiving surface contacting the supply surface;
wherein a length of the supply surface in the opening and closing direction is larger than a length of the output surface in a width direction intersecting with the opening and closing direction.

2. The medical treatment tool according to claim 1, wherein the electric heater has a top surface intersecting with the supply surface, the top surface is offset in the opening and closing direction from the output surface.

3. The medical treatment tool according to claim 1, wherein the output surface is flat relative to a virtual line perpendicular to the opening and closing direction.

4. The medical treatment tool according to claim 3, wherein the output surface is perpendicular to the supply surface.

5. The medical treatment tool according to claim 1, wherein the first jaw further comprises a wiring configured to supply electrical energy to the electric heater, the electric heater has a receiving surface opposed to the supply surface, and the receiving surface contacts the wiring.

6. The medical treatment tool according to claim 1, wherein the first jaw further comprises a first electrode, the second blade comprises a second electrode, a first current flow is made between the first electrode and the second electrode.

7. The medical treatment tool according to claim 6, wherein the first electrode is the first blade.

8. The medical treatment tool according to claim 7, wherein the first jaw further comprises a third electrode, the third electrode is spaced apart from the first electrode in the width direction, a second current flow is made between the first electrode and the third electrode.

9. The medical treatment tool according to claim 7, wherein the first jaw further comprises a thermoelectrical insulator and a wiring configured to supply electrical energy to the first electrode, the wiring located between the first electrode and the thermoelectrical insulator.

10. The medical treatment tool according to claim 1, wherein the first jaw further comprises a thermoelectrical insulator.

11. The medical treatment tool according to claim 10, wherein the thermoelectrical insulator has a side wall and a bottom surface, the side wall contacts a first surface of the first electrode opposed to the heat receiving surface, the bottom wall contacts a second surface of the first electrode opposed to the output surface, a thickness of the side wall in the width direction is larger than a thickness of the bottom wall in the opening and closing direction.

12. The medical treatment tool according to claim 10, wherein the thermoelectrical insulator has a side wall and a bottom surface, the side wall contacts a first surface of the heater opposed to the supply surface, the bottom wall contacts a second surface of the first electrode opposed to the output surface, a thickness of the side wall in the width direction is larger than a thickness of the bottom wall in the opening and closing direction.

13. The medical treatment tool according to claim 10, wherein the thermoelectrical insulator sandwiches the output surface and the electric heater in the width direction.

14. The medical treatment tool according to claim 13, wherein the electric heater has a top surface intersecting with the supply surface and a side surface opposed to the supply surface, the top surface and the side surface are covered by the thermoelectrical insulator.

15. The medical treatment tool according to claim 14, wherein the electric heater has a bottom surface opposed to the top surface, the bottom surface is covered by the thermoelectrical insulator.

16. The medical treatment tool according to claim 10, wherein the thermoelectrical insulator is made by a resin.

17. The medical treatment tool according to claim 1, wherein the heat receiving surface is inclined relative to the opening and closing direction.

18. The medical treatment tool according to claim 1, wherein the heat receiving surface intersects with the output surface.

19. The medical treatment tool according to claim 1, wherein the output surface is concave relative to a virtual line perpendicular to the opening and closing direction.

20. The medical treatment tool according to claim 19, wherein the output surface is curved.
